(19) United States
(12) Plant Patent Application Publication
(10) Pub. No.: US 2014/0223625 P1
(43) Pub. Date: Aug. 7, 2014

(54) Mint plant "Canyon Mint"

(50) ;

(71) Applicant: ESSEX LABORATORIES, INC., Napavine, WA (US)

(72) Inventor: Donald D. Roberts, Independence, OR (US)

(73) Assignee: ESSEX LABORATORIES, INC., Napavine, WA (US)

(21) Appl. No.: 13/815,164

(22) Filed: Feb. 4, 2013

(51) Int. Cl. A01H 5/00

(52) U.S. Cl. PLT/259

(57) ABSTRACT

Mint selection 05-19-1 denominated “Canyon Mint” is a new Mentha piperita cultivar that produces an essential oil different in composition than commercially grown mint varieties. The essential oil is similar to standard mint oil in components composition but differs in the typical ratio of components. Organoleptically, it differs from typical peppermint oil. It is resistant to mint rust and more resistant to Verticillium mint wilt than current commercially grown varieties.

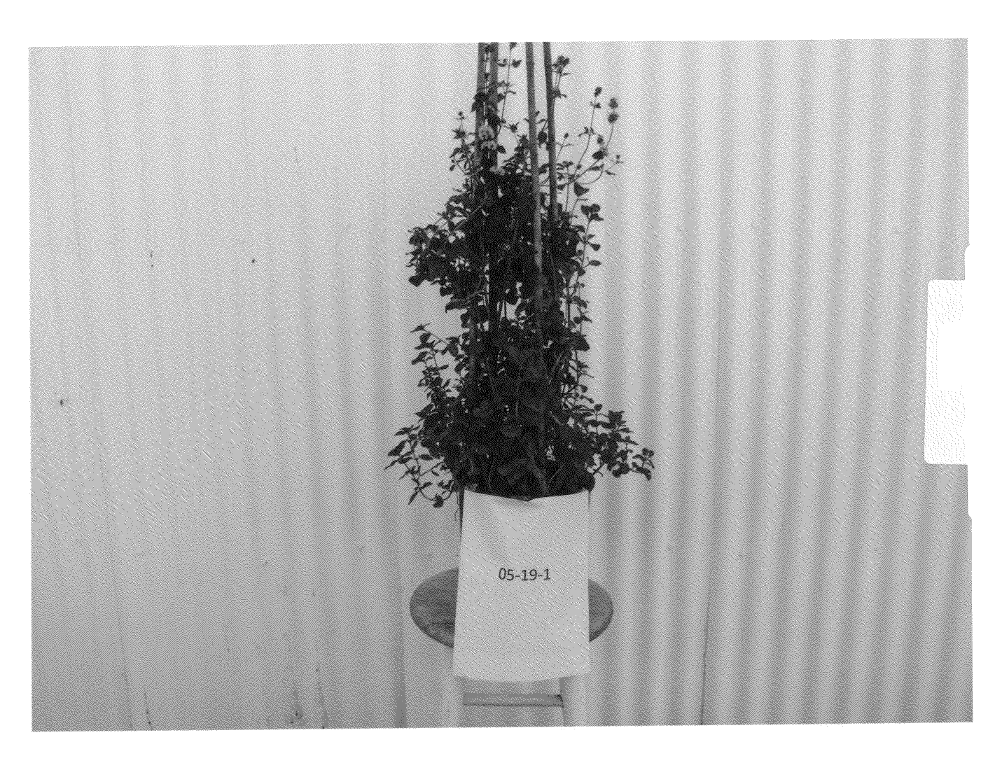

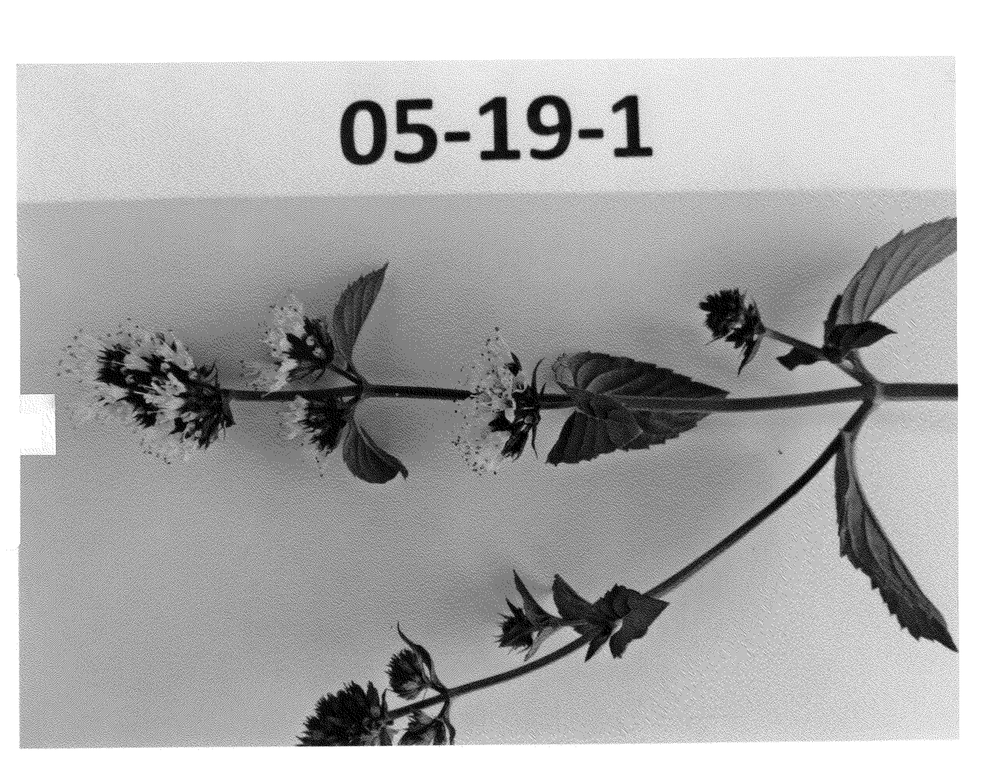

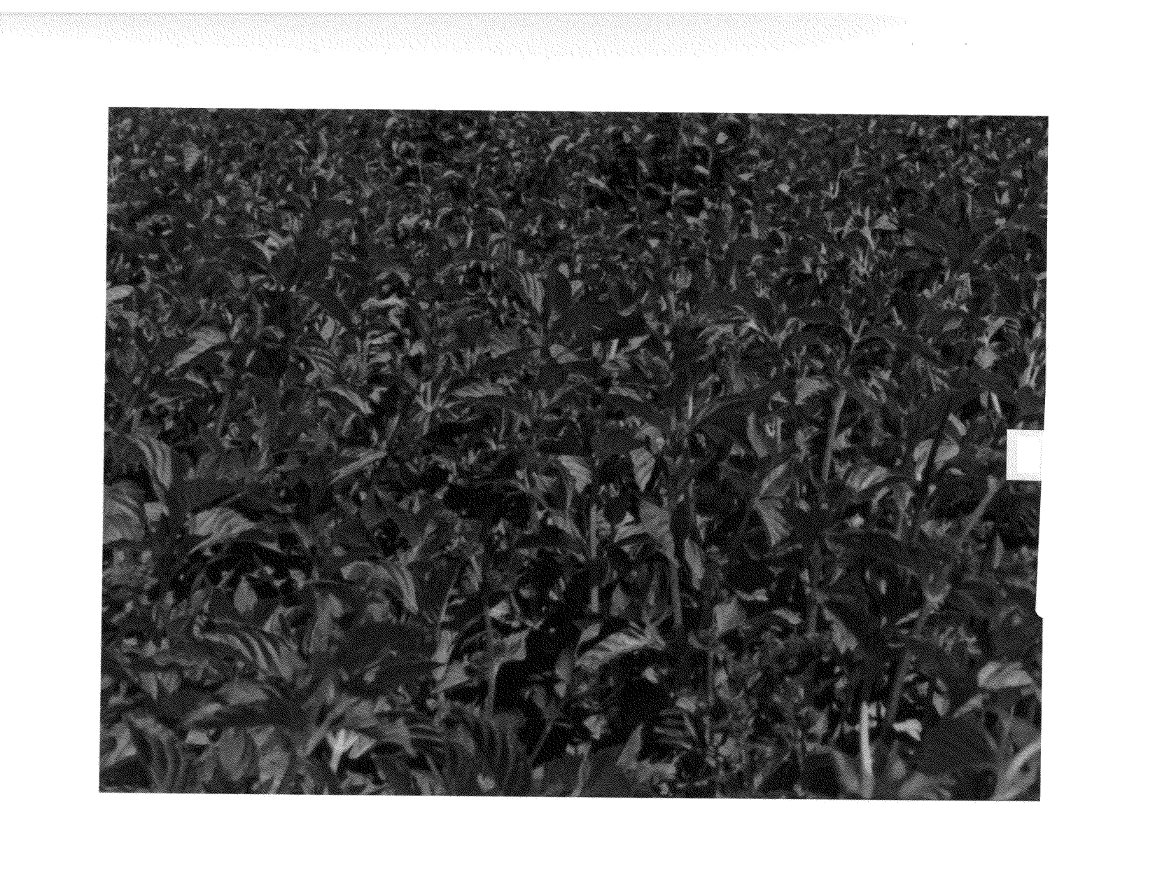

### FEDERAL SPONSORSHIP

None

### TYPE OF PLANT AND NAME OF VARIETY

The present invention relates to a new and distinct variety of peppermint plant developed from a parent of the species Mentha piperita L. The new variety will be identified as “Canyon Mint.”

### SUMMARY OF THE INVENTION

Mint selection 05-19-1 denominated “Canyon Mint” is a new Mentha piperita cultivar that produces an essential oil different in composition than commercially grown mint varieties. The essential oil is similar to standard mint oil in components composition but differs in the typical ratio of components. Organoleptically, it differs from typical peppermint oil. It is resistant to mint rust and more resistant to Verticillium mint wilt than current commercially grown varieties.

### DISCOVERY AND ASEXUAL REPRODUCTION

This new peppermint was developed in a mint breeding program in which the primary objective was to develop a Mitcham type peppermint variety having a specific oil composition, acceptable yield and resistant to diseases mint wilt and mint rust. Mint wilt is caused by a soil-borne fungus, Verticilium dahlia and mint rust is a leaf infection caused by the air-borne fungus Puccinia menthae. Only seedlings with no symptoms of mint wilt or rust were selected for further evaluation. Selection 05-19-1 was vegetatively propagated to increase the selection to 20 plants for continued evaluation in 2006. The selection was again vegetatively propagated to over 300 plants for planting and evaluation in 2007. When compared to Black Mitcham as a control, 05-19-1 had less (or no) symptoms of mint wilt than the control and had no mint rust infection. Yield of 05-19-1 is equivalent to Black Mitcham in test plots as a single plant and as multiple plants in larger plots.

Selection 05-19-1 originated as a seedling from an open pollinated polyploid ‘Black Mitcham’ parent. Diploid Black Mitcham peppermint is sterile and only becomes fertile in the polyploidy state. The Black Mitcham parent to 05-19-1 was one of several mint lines in a polycross breeding system composed of selected male and female fertile genotypes.

Selection 05-19-1 is asexually propagated to maintain the cultivar's genetic integrity and as a means of increasing the selection for commercial planting. Asexual propagation, by tip cuttings or stolon sections, is a common practice in commercial mint cultivation and serves as a means of propagating the normally sterile mint plant. Under the inventor's direction, Premier Botanicals has conducted asexual propagation of 05-19-1 for greenhouse and field planting in Monmouth, Oreg., each year since 2005 and the genotype comes true to form with each generation.

### BRIEF DESCRIPTION OF THE PHOTOGRAPHS

The accompanying color photographs show typical greenhouse and field grown vegetative growth of 05-19-1 and depicts the color as nearly true as reasonably possible.

Photographs 1 and 2 illustrate the flowering pattern and multiple flowering shoots of my new mint plant in accordance with the present invention. Photograph 2 illustrates the shape of the flower collected from a field grown plant.

Photographs 2 and 3 illustrate the leaf shape of my new mint plant. Photograph 3 illustrates the shape of the leaf and compact growth of 05-19-1 grown under field conditions.

### DESCRIPTION OF PLANT

My new mint plant improves upon and is distinct from other mint plants in several characteristics, including but not limited to, the following:

1. The ability to produce an essential oil different in composition but with similar components as ‘Black Mitcham’ peppermint;

2. A more compact plant but with a branching pattern similar to its parent;

3. An early spring growth similar to Black Mitcham peppermint but with an earlier maturity for desirable chemical composition of its essential oil;

4. A level of resistance to mint wilt (Verticillium dahliae) equal to or greater than that of its parent;

5. Resistance to mint rust caused by Puccinia menthae.

The essential oil extracted from 05-19-1 has the same components as that of commercial oil produced by Black Mitcham peppermint as illustrated in Table 1. However, the ratio of oil components differs between the commercial peppermint oil and that of 05-19-1. The concentration of menthone and menthol in the oil of 05-19-1 is lower than that of Black Mitcham. The amount of Menthofuran and Menthyl Acetate in the oil of 05-19-1 is higher than that of Black Mitcham. Organoleptically, the oil of 05-19-1 is different from that of Black Mitcham, reflecting the difference in oil component ratios.

TABLE 1
Composition of 05-19-1 Peppermint Oil from Test Plots Compared to
commercial Black Mitcham Peppermint Oil and Oil from Black Mitcham
Peppermint Grown in Test Plots located near Monmouth, Oregon.

| | Commercial | 2005 Test Plot | 2005 Test Plot |
|---|---|---|---|
| | Black Mitcham 1/ | Black Mitcham 2/ | 05-19-1 3 |
| α-pinene | 0.82 | 0.80 | 0.84 |
| β-pinene | 1.06 | 1.03 | 1.17 |
| 1-limonene | 1.78 | 1.73 | 2.24 |
| 1,8-cineole | 5.33 | 4.90 | 3.71 |
| 1-menthone | 20.00 | 19.43 | 2.47 |
| Menthofuran | 4.35 | 4.23 | 44.02 |
| Iso-menthone | 3.19 | 3.14 | 0.58 |
| 1-menthyl acetate | 5.09 | 5.36 | 19.67 |
| Neo-menthol | 5.40 | 5.52 | 1.90 |
| B-caryophyllene | 0.83 | 0.85 | 1.60 |
| 1-menthol | 45.20 | 44.6 | 18.3 |
| Pulegone | 2.17 | 2.14 | <1.0 |
| Germacrene-D | 1.22 | 1.37 | <1.0 |

The numbers listed in the above table are percentages based upon the analysis of the respective mint oils by gas chromatography. The percentages are determined by calculation of the relative peak areas.
1/ Commercial oil sample typical of what is produced by peppermint producers.
2/ Black Mitcham oil from a control plant in the 2005 seedling test plot.
3 05-19-1 oil from a single plant harvest from the 2005 test plots.

Taxonomic Description of 05-19-1

This new plant, under greenhouse and field growing conditions, is a bush type plant with lateral branches at each node of the main stems. The height of 05-19-1 is slightly less than Black Mitcham growing under similar conditions and will vary based on fertilizer, soil quality, and water application, amongst other known factors that affect growth patterns. Secondary and tertiary branching occurs to form a compact growth habit. When 05-19-1 is mature and ready for harvest, the main stem at mid-plant (approximately between the eleventh and twelfth node) is 3.9-4.2 mm in width. The secondary and tertiary branch stems are 1.5 -2 mm and 1-2 mm in width, respectively.

Mature leaves at the bottom of the plant are ovate lanceolate as are leaves on secondary branch stems. Leaves on upper mature plants, both main and secondary stems, are more lanceolate (Photograph 3). Mid-main stem leaf size at flowering is 28-32 mm in width and 58-65 mm in length. Leaf size on secondary branches at flowering is 16-20 mm in width and 28-32 mm in length. Leaf petioles on the main stem leaves are 10-14 mm in length while petioles on secondary branch stem leaves are 4-6 mm in length. Leaves on the mid-main stem and lower tend to be slightly lobed and denticulate while the leaves on the upper plant tend to be more dentate. The main stem leaves have from 6 to 10 teeth on each side. The leaf is dark green in color, ranging from Royal Horticulture Society Color Code 137B to 137C in the green group classification. The leaf has 6-9 lateral veins, more or less in parallel off the main vein that runs from the petiole to the tip of the leaf. The veins are prominent in all leaves of 05-19-1.

The inflorescence is an inconspicuous spike with capitate flowers developing at the last few nodes of the stem. The capitate flowers are 15-20 mm in width and 10-15 mm in length. The flowers consist of five petals fused into a two lipped corolla. The corolla is light in color ranging from Royal Horticulture Society Color Code 84D to white in the Violet Group The Calyx is generally yellow-green and is 143C RHS Color Code, Green Group. The gynoecium consists of a single pistil with two lobed stigma that is exserted. The androecium consists of four stamens, each with a distinct filament and anther.

While the plant that comprises the present invention has been described in connection with a specific embodiment thereof, it will be understood that this application is intended to cover any variation, uses, or adaptation of the invention (particular those induced by cultivation under different environmental conditions) following, in general, the principles of the invention and including such departures from the present disclosure as come within known or customary practice in the art to which the invention pertains and as may be applied to the essential features hereinbefore set forth, and as fall within the scope of the invention and the limits of the appended claim.

I claim:

1. A new and distinct variety of peppermint plant, substantially as shown and described, characterized particularly by improving resistance to mint rust, and producing a unique essential oil.
